(19) United States
(12) Patent Application Publication
(10) Pub. No.: US 2026/0018266 A1
(43) Pub. Date: Jan. 15, 2026

(54) Methods and Systems for Streaming, Managing, and Utilizing Multimodal Wellness Data

(71) Applicant: The Prudential Insurance Company of America, Newark, NJ (US)

(72) Inventors: Vivian Muniz, São Paulo (BR); Marcelo Fonseca, São Paulo (BR); Thiago Monaco, São Paulo (BR)

(21) Appl. No.: 19/250,738

(22) Filed: Jun. 26, 2025

Related U.S. Application Data
(60) Provisional application No. 63/671,600, filed on Jul. 15, 2024.

(51) Int. Cl. G16H 10/60; G06F 16/215; G16H 20/00

(52) U.S. Cl. CPC G16H 10/60; G06F 16/215; G16H 20/00

(57) ABSTRACT

This application is directed to adaptively managing holistic wellness of multiple users via a cloud-based multimodal personal data management platform. A computer device executing a multimodal wellness application obtains an aggregation of data related to a user from two or more data pools. At least one of the data pools is hosted by a third-party application, distinct from the multimodal wellness application. The computing device associates the aggregation with different wellness domains to determine a multimodal wellness score indicating a quality of an association of the data with the plurality of wellness domains. Based on the multimodal wellness score, the computing device determines a set of data rules associated with one or more of the plurality of wellness domains. And the computing device displays one or more user interface elements indicating a progression towards an objective based on satisfaction of respective data rules of the set of data rules.

### RELATED APPLICATION

This application claims priority to U.S. Provisional Patent Application No. 63/671,600, entitled “Methods and Systems for Streaming, Managing, and Utilizing Multimodal Wellness Data,” filed Jul. 15, 2024, which is hereby incorporated by reference in its entirety.

### TECHNICAL FIELD

This application relates generally to data management technology, and specifically to systems, methods, and non-transitory computer-readable storage medium for dynamically and holistically managing large amount of multimodal data on a cloud-based data management platform.

### BACKGROUND

Digital content is rapidly gaining popularity, including digital content related to optimizing wellness of individuals based on capabilities of the individuals' electronic devices to coordinate, for example, sensor data and notifications. However, many current solutions do not provide holistic wellness for users, and instead only provide wellness-related content that is relevant to a particular wellness domain, or an otherwise non-comprehensive subset of wellness domains that must be considered to optimize holistic wellness most efficiently and intuitively.

### SUMMARY

Various embodiments of this application are directed to methods, systems, devices, and non-transitory computer-readable storage media for adaptively managing a plurality of users' holistic wellness by aggregating multimodal data from a plurality of different sources (e.g., data pools), including data that is managed, hosted, and streamed by disparate third parties. A multimodal wellness platform consolidates a plurality of data associated with the different sources to draw inferences (e.g., through use of artificial intelligence (AI)) about a user's overall wellness condition based on relationships determined by aggregating the data from the plurality of data pools.

Some embodiments described herein provide users with personalized information for improving well-being based on an interplay (e.g., data fusion) of data from a plurality of different data sources. Some embodiments described herein implement a wellness balance that is dynamically updated based on goals completed and activities performed in the application. Some embodiments described herein including monitoring an individualized wellness balance configured to indicate user progress and drive behavioral change.

In some embodiments, a dynamic scoring algorithm is applied to provide adaptive and multifaceted assessment of individual wellness, covering different data items associated with physical, mental, and financial health. The dynamic scoring algorithm may utilize machine learning, incremental data fetching, advanced analytics, or a combination thereof to continually update and personalize wellness scores across multiple wellness domains. The embodiments described herein provide a multimodal and quantitative view of a user's life, considering variables from income and debt to physical fitness levels and mental resilience.

This comprehensive model integrates various wellness factors, such as financial stability, emergency preparedness, and mental well-being, into a single, dynamic score. By doing so, it offers a more holistic understanding of wellness, adaptable to the unique and changing circumstances of each individual. This makes it an innovative solution for a total wellness ecosystem.

In accordance with some embodiments, an example method is provided. The example method includes, at a computing device having a display and one or more processors, executing a multimodal wellness application. The example method includes obtaining an aggregation of data related to a user from two or more data pools, where at least one respective data pool of the two or more data pools is hosted by a third-party application, distinct from the multimodal wellness application. The example method includes associating the obtained aggregation of the data related to the user with a plurality of wellness domains. The example method includes, in accordance with associating the data related to the user of the obtained aggregation with the plurality of wellness domains, determining a multimodal wellness score indicating a quality of an association of the data with the plurality of wellness domains. The example method includes, based on the multimodal wellness score, determining a set of data rules associated with one or more of the plurality of wellness domains. The example method includes displaying, at the display of the computing device, one or more user interface elements indicating a progression towards an objective, where the progression is based on satisfaction of respective data rules of the set of data rules.

In another aspect, some implementations include a computer system that includes one or more processors and memory having instructions stored thereon, which when executed by the one or more processors cause the processors to perform any of the above methods.

In yet another aspect, some implementations include a non-transitory computer-readable medium, having instructions stored thereon, which when executed by one or more processors cause the processors to perform any of the above methods.

These illustrative embodiments and implementations are mentioned not to limit or define the disclosure, but to provide examples to aid understanding thereof. Additional embodiments are discussed in the Detailed Description, and further description is provided there.

### BRIEF DESCRIPTION OF THE DRAWINGS

For a better understanding of the various described implementations, reference should be made to the Detailed Description below, in conjunction with the following drawings in which like reference numerals refer to corresponding parts throughout the figures.

FIG. 1 is a diagram of an example computing system for managing multimodal personal data associated with holistic wellness of users, in accordance with some embodiments.

FIG. 2A is a block diagram illustrating a server system configured for a multimodal wellness data management system, in accordance with some embodiments.

FIG. 2B is a block diagram illustrating a computing device configured to execute a multimodal wellness application, in accordance with some embodiments.

FIGS. 3A to 3G illustrate example user interfaces of a multimodal wellness application, in accordance with some embodiments.

FIGS. 4A to 4C illustrate example user interfaces for interacting with a wellness-domain-specific sub-application of a multimodal wellness application, in accordance with some embodiments.

FIG. 5 illustrates a flow diagram of an example method for managing data, in accordance with some embodiments.

Like reference numerals refer to corresponding parts throughout the several views of the drawings.

### DETAILED DESCRIPTION

Reference will now be made in detail to specific embodiments, examples of which are illustrated in the accompanying drawings. In the following detailed description, numerous non-limiting specific details are set forth in order to assist in understanding the subject matter presented herein. But it will be apparent to one of ordinary skill in the art that various alternatives may be used without departing from the scope of claims and the subject matter may be practiced without these specific details. For example, it will be apparent to one of ordinary skill in the art that the subject matter presented herein can be implemented on many types of electronic devices with digital video capabilities.

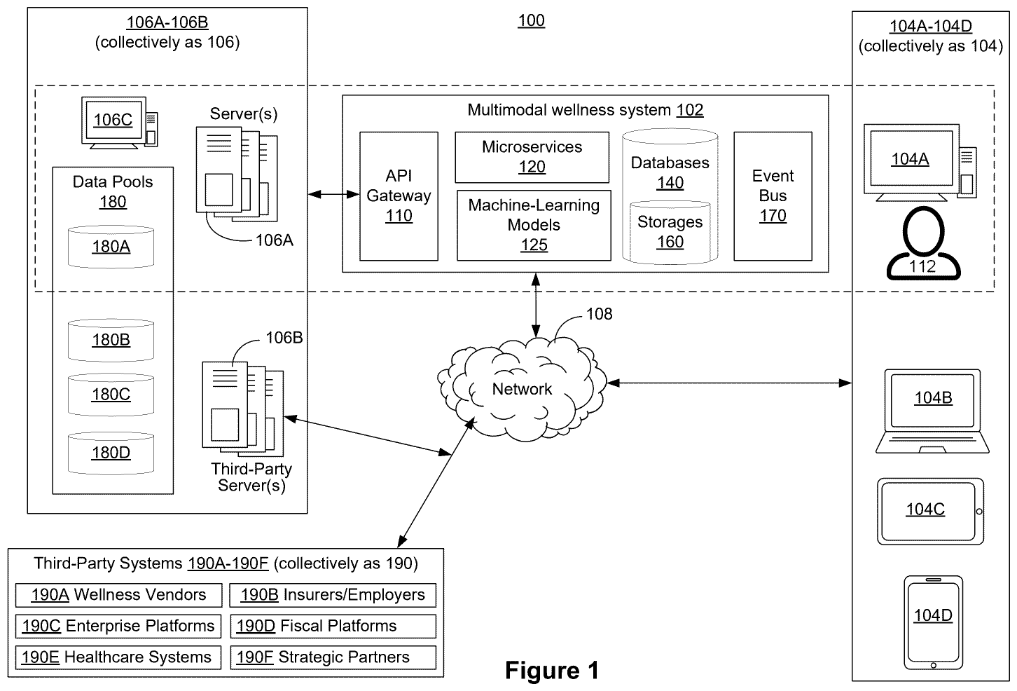

FIG. 1 is a diagram of an example computing system 100 for managing multimodal personal data associated with holistic wellness of users 112, in accordance with some embodiments. The computing system 100 includes one or more servers 106 communicatively coupled to one or more computing devices 104, and enables a cloud-based data management platform that hosts a plurality of user accounts associated with the users 112. In some embodiments, a multimodal wellness application is executed by the computing system 100, and each user 112 accesses the multimodal wellness application via a respective computing device 104. The computing system 100 may manage large amount of data associated with a large number of users and collected from multiple distributed third parties, while ensuring timeliness and security of the associated data. Management of such a complicated system involving so many players requires technology solutions (e.g., data pooling, data security measures, communication traffic control, API integration, multimodal data integration) that are significantly more than conventional computer functions.

The computing system 100 includes a multimodal wellness system 102 for facilitating managing holistic wellness of a user 112 based on multimodal personal data, in accordance with some embodiments. In some embodiments, the multimodal wellness system 102 is located entirely on the computing device 104. In some embodiments, portions of the multimodal wellness system 102 are located on a remote server (e.g., application server 106A discussed below).

In some embodiments, the multimodal wellness system 102 includes an API gateway 110, which may be used to facilitate communication between one or more computing device(s) 104 and the one or more server(s) 106, and/or to facilitate communication between two or more of the servers 106. In some embodiments, the API gateway can be used to facilitate communications between one or more third-party servers 106B and a computing device 104.

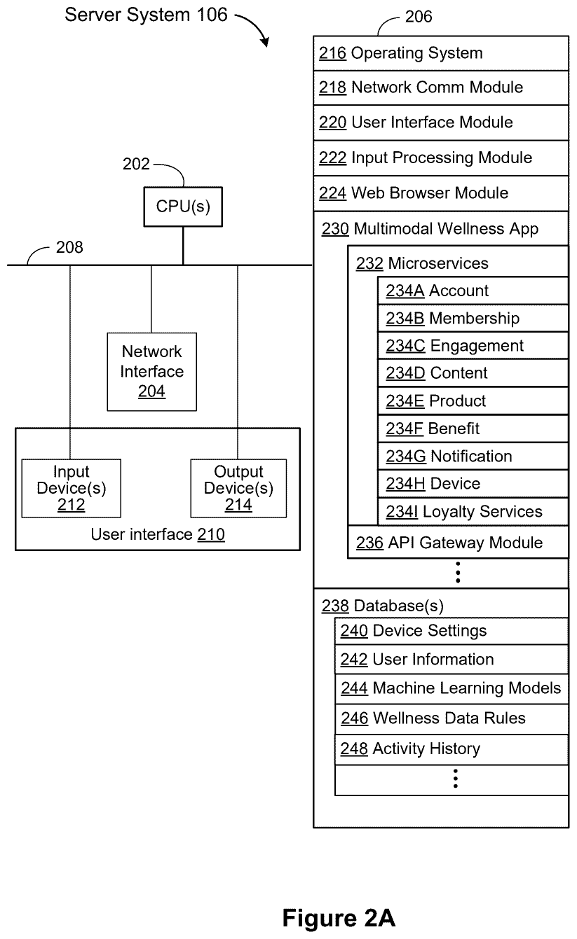

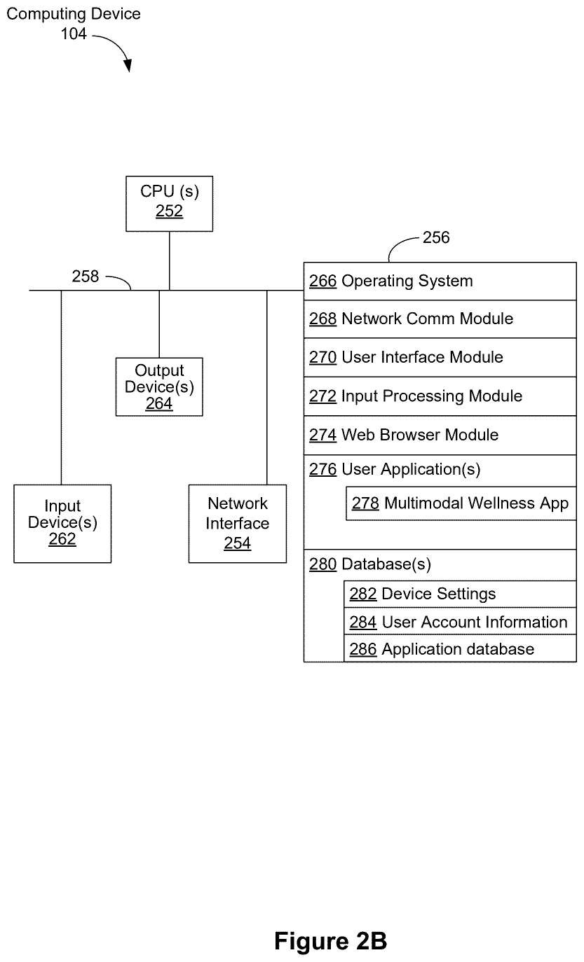

In some embodiments, the multimodal wellness system 102 includes one or more microservices 120, which can be configured to perform tasks that facilitate management of holistic wellness of the user 112. Examples of individual microservices of the multimodal wellness system 102 are described below with respect to FIG. 2A.

In some embodiments, the multimodal wellness system includes one or more databases 140 and one or more storages 160, which may be configured to store data related to the multimodal wellness system 102. For example, a respective database of the databases 140 may include wellness data of the user 112 (e.g., historic multimodal wellness, wellness-domain-specific wellness scores, aggregations of data related to the user 112 that are combined from two or more of the data pools 180).

In some embodiments, the multimodal wellness system 102 includes an event bus 170 for controlling flow of events across the multimodal wellness system 102. For example, the event bus 170 can obtain user inputs provided by the user 112 to cause requests to the application server 106A and/or one or more of the third-party servers 106B across the API gateway 110.

The one or more computing devices 104 may be, for example, desktop computers 104A, laptop computers 104B, tablet computers 104C, mobile phones 104D, or any other computing devices. Each computing device 104 can collect data or user inputs, executes user applications, and present outputs on its user interface. In accordance with some embodiments, a computing device (e.g., desktop computer 104A) of the one or more computing devices 104 can be used for a user 112 to access a multimodal wellness application, which may be provided by the multimodal wellness system 102.

The one or more servers 106 can includes application servers 106A associated with the multimodal wellness system 102. In some embodiments, an administrative server 106C (e.g., a device connectable to the server environment) is configured to manage aspects of the multimodal wellness application 102. The collected data or user inputs can be processed locally at the computing device 104 and/or remotely by the server(s) 102.

The one or more servers 106 can provide system data (e.g., boot files, operating system images, and user applications) to the computing devices 104, and in some embodiments, process the data and user inputs received from the computing device(s) 104 when the user applications are executed on the computing devices 104.

The one or more servers 106 are configured to enable real-time data communication with the computing devices 104 that are remote from each other or from the one or more servers 106. Further, in some embodiments, the one or more servers 106 are configured to implement data processing tasks that cannot be or are preferably not completed locally by the computing devices 104. For example, the computing devices 104 include a laptop computer 104B that executes an information management application for tracking and visualizing a plurality of metrics associated with a project. The one or more servers 106 collects historic data and current data concerning the project or other related projects from one or more data sources. The historic data and current data are consolidated, processed, and visualized interactively in real time. For example, such data are matched with other data, categorized, or applied to synthesize related data (e.g., predict a predicted performance trend, generate an alert message). In some embodiments, historic data and current data are provided by multiple data sources, have unclear or weak correlations, and include natural language data collected from individual users 112 in a subjective and descriptive format. Stated another way, a server system may include a plurality of servers 106 (e.g., a host server 106A and alternative servers 106B) configured to create an information management platform to collect, process, and visualize a large volume of complex data, which cannot be accomplished by human.

The one or more servers 106, one or more computing devices 104, and the multimodal wellness system 102 are communicatively coupled to each other via one or more communication networks 108, which are the medium used to provide communications links between these devices and computers connected together within the computing system 100. The one or more communication networks 108 may include connections, such as wire, wireless communication links, or fiber optic cables. Examples of the one or more communication networks 108 include local area networks (LAN), wide area networks (WAN) such as the Internet, or a combination thereof.

The one or more communication networks 108 are, optionally, implemented using any known network protocol, including various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 108 may be established either directly (e.g., using 3G/4G connectivity to a wireless carrier), or through a network interface (e.g., a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational, and other computer systems that route data and messages.

In some embodiments, both model training and data inference are implemented locally at each computing device 104. The computing device 104 obtains the training data from the one or more servers 106, applies the training data to train the machine learning models, and uses the learning models to process current data. Alternatively, in some embodiments, data inference is implemented locally at a computing device 104, while model training is implemented remotely at a server 106 associated with the computing device 104. The server 106B obtains the training data from itself, another server 106 or the storage 160 and applies the training data to train the machine learning models. The trained machine learning models are optionally stored in the server 106B or storage 160. The computing device 104 imports the trained machine learning models from the server 106B, the database(s) 140, and/or the storage(s) 160, processes the current data using the machine learning models, and generates data processing results (e.g., a performance projection trend, an alert message) to be presented on a user interface.

In some embodiments, a machine-learning model 125 of the multimodal wellness application 102 applies deep learning techniques that collects, processes, and visualizes data associated with a project. In some embodiments, in these deep learning techniques, machine learning models (e.g., performance projection model) are created based on one or more neural networks to process the data. A machine learning model is trained with training data (e.g., historic data) before they are applied to process current data that are collected in real time for data inference. Further, in some embodiments, the machine learning model is further trained using the current data.

Alternatively, in some embodiments, both model training and data inference are implemented remotely at a server 106 (e.g., the server 106A) associated with a computing device 104, particularly if a large volume of complex data are involved. The server 106A obtains the training data from itself, another server 106 or the storage 160 and applies the training data to train the machine learning models. The computing device 104 receives data processing results from the server 106A and presents the results on a user interface. The computing device 104 itself implements no or little data processing on the data processing results. In some embodiments, the computing device 104 enters an input to define one or more parameters (e.g., a target projection length) for data inference, and the server 106 generates the data processing results based on the input. Additionally, in some embodiments, a client-side information management application collaborates with a server-side information management application to deliver the data processing results. The client-side information management application presents a user interface where the input from a user is received, and data processing results are presented. The server-side information management application collects and processes data (e.g., using the deep learning techniques), and enables display of the user interface.

In accordance with some embodiments, the computing system 100 includes a plurality of data pools 180, from which the multimodal wellness system 102 can obtain one or more aggregations of data of the user 112 for determining and/or updating wellness scores. In some embodiments, one or more data pools (e.g., a data pool 180A) are associated with the multimodal wellness system 102 (e.g., stored on the application server 106A). In some embodiments one or more of the data pools (e.g., data pool 180B, data pool 180C, and/or data pool 180D) are associated with (e.g., hosted on) one or more of the third-party servers 106B. For example, the data pool 180B may be associated with a healthcare provider (e.g., associated with the healthcare system 190E). In some embodiments, the multimodal wellness system applies rate limits to requests to the data pools 180 (e.g., based on respective throttling techniques of the respective third-party systems 190 controlling access to the respective data pools 180 that are accessed).

In accordance with some embodiments, the multimodal wellness system 102 is configured to communicate with one or more third-party systems 190 (e.g., over the API gateway 110). Examples of third parties of the one or more third-party systems 190 can include:
Wellness vendors 190A, including meditative content providers, fiscal savings content providers, mental activity content providers, etc.;
Insurers/Employers 190B, including insurers and/or employers of the users 112 112, who may be configured to provide access of the multimodal wellness application 102 to the user 112;
Enterprise platforms 190C, including data management providers, cloud-computing providers, AI inference providers;
Fiscal platforms 190D, including, for example, banking institutions, credit unions, and other platforms having access to users 112′ fiscal data;
Healthcare systems 190E, including, for example, healthcare providers, health-based electronic devices, such as smart watch providers, fitness equipment providers, etc.;
Strategic partners 190F, including, for example, systems configured to facilitate wellness for particular wellness domains;

In some embodiments, one or more of the third-party systems 190 are associated with one or more of the data pools 180. In some embodiments, the third-party systems are configured to require access authorization for the user to use applications and/or data from the third-party systems 190. In some embodiments, the multimodal wellness system 102 is configured to store authentication information for users 112′ access to the data pools and/or other aspects of the third-party systems, such that the user's access to the third parties can be automatically managed by the multimodal wellness system 102.

In some embodiments, additional means of access authorization are provided for accessing aspects of the multimodal wellness application 102, such that a high level of security is provided for data that may be sensitive to the user 112 (e.g., respective aggregations of data related to the user 112 combined from the data pools 180).

FIG. 2A is a block diagram illustrating a server system configured for a multimodal wellness data management system, in accordance with some embodiments. The server system 106 includes an application server 106A. The server system 106 typically includes one or more processing units (CPUs) 202, one or more network interfaces 204, memory 206, and one or more communication buses 208 for interconnecting these components (sometimes called a chipset). In some embodiments, the server system 106 includes a user interface system 210 that further includes one or more input devices 212 that facilitate user input or one or more output devices 214 that enable presentation of user interfaces and display content.

Memory 206 includes high-speed random-access memory, such as DRAM, SRAM, DDR RAM, or other random-access solid-state memory devices; and, optionally, includes non-volatile memory, such as one or more magnetic disk storage devices, one or more optical disk storage devices, one or more flash memory devices, or one or more other non-volatile solid state storage devices. Memory 206, optionally, includes one or more storage devices remotely located from one or more processing units 202. Memory 206, or alternatively the non-volatile memory within memory 206, includes a non-transitory computer readable storage medium.

In some embodiments, memory 206, or the non-transitory computer readable storage medium of memory 206, stores the following programs, modules, and data structures, or a subset or superset thereof:
Operating system 216 including procedures for handling various basic system services and for performing hardware dependent tasks;
Network communication module 218 for connecting each server 106 to other devices (e.g., server 106, computing device 104, or storage 160) via one or more network interfaces 204 (wired or wireless) and one or more communication networks 108, such as the Internet, other wide area networks, local area networks, metropolitan area networks, and so on;
User interface module 220 for enabling presentation of information (e.g., a graphical user interface for an application 206, widgets, websites, and web pages thereof, and/or games, audio and/or video content, text, etc.) at each computing device 104 via one or more output devices 214 (e.g., displays, speakers, etc.);
Input processing module 222 for detecting one or more user inputs or interactions from one of the one or more input devices 212 and interpreting the detected input or interaction;
Web browser module 224 for navigating, requesting (e.g., via HTTP), and displaying websites and web pages thereof;
Server-side multimodal wellness application 230 for execution by the server system 106A to coordinate operations of a client-side device that the user 112 is accessing from the computing device 104, where the server-side information management application 206 further includes one or more of microservices 232 for aspects of performance of the server-side multimodal wellness application 230, including: (i) an account microservice 234A; (ii) a membership microservice 234B; (iii) an engagement microservice 234C; (iv) a content microservice 234D; (v) a product microservice 234E; (vi) a benefit microservice 234F; (vii) a notification microservice 234G; (viii) a device microservice 234H; and/or (ix) a loyalty services microservice 234I;
One or more databases 238 for storing at least data including one or more of:
   Device settings 240 including common device settings (e.g., service tier, device model, storage capacity, processing capabilities, communication capabilities, etc.) of a server 106;
   o User account information 242 for the multimodal wellness application 230, e.g., user names, security questions, account history data, user preferences, and predefined account settings, wellness scores, activity history;
   Machine learning model(s) 244 for processing data (e.g., aggregated data from two or more of the data pools 180);
   Wellness data rules 246 determined for users 112 based on their respective multimodal wellness scores; and
   Activity history 248 for storing historic data related to, for example, users 112′ multimodal data scores, activities performed by the user as part of the determined data rules, etc.

Optionally, each of the one or more databases 238 is stored in one of the host server 106A, alternative servers 106B, and storage 160 of the multimodal wellness system 102. Optionally, the one or more databases 238 are distributed in more than one of the server 106A, alternative servers 106B, and storage 160 of the multimodal wellness system 102. In some embodiments, more than one copy of the above data is stored at distinct devices, e.g., two copies of the machine learning model 244 are stored at the application server 106A and local storage 160 at the computing device 104, respectively.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, memory 206, optionally, stores a subset of the modules and data structures identified above. Furthermore, memory 206, optionally, stores additional modules and data structures not described above.

FIG. 2B is a block diagram illustrating a computing device 104 configured to execute a multimodal wellness application 278, in accordance with some embodiments. The computing device 104 typically includes one or more processing units (CPUs) 252, one or more network interfaces 254, memory 256, and one or more communication buses 258 for interconnecting these components (sometimes called a chipset). The computing device 104 includes one or more input devices 262 that facilitate user input or one or more output devices 264 that enable presentation of user interfaces and display content.

Memory 256 includes high-speed random-access memory, such as DRAM, SRAM, DDR RAM, or other random-access solid-state memory devices; and, optionally, includes non-volatile memory, such as one or more magnetic disk storage devices, one or more optical disk storage devices, one or more flash memory devices, or one or more other non-volatile solid state storage devices. Memory 256, optionally, includes one or more storage devices remotely located from one or more processing units 252. Memory 256, or alternatively the non-volatile memory within memory 256, includes a non-transitory computer readable storage medium. In some embodiments, memory 256, or the non-transitory computer readable storage medium of memory 256, stores the following programs, modules, and data structures, or a subset or superset thereof:
Operating system 266 including procedures for handling various basic system services and for performing hardware dependent tasks;
Network communication module 268 for connecting each computing device 104 to other devices (e.g., server 106, computing device 104, or storage 160) via one or more network interfaces 254 (wired or wireless) and one or more communication networks 108;
User interface module 270 for enabling presentation of information at each computing device 104 via one or more output devices 264 (e.g., displays, speakers, etc.);
Input processing module 272 for detecting one or more user inputs or interactions from one of the one or more input devices 262 and interpreting the detected input or interaction;
Web browser module 274 for navigating, requesting (e.g., via HTTP), and displaying websites and web pages thereof;
One or more user applications 276 for execution by the computing device 104 (e.g., games, social network applications, smart home applications, and/or other web or non-web-based applications), where in some embodiments, the user application(s) 276 include a client-side multimodal wellness application 278 for execution by the computing device 104 to visualize data associated with a plurality of metrics of one or more projects;
One or more databases 280 for storing at least data including one or more of:
   Device settings 282 including common device settings (e.g., service tier, device model, storage capacity, processing capabilities, communication capabilities, etc.) of a server 106;
   User account information 284 for the information management application 206, e.g., user names, security questions, account history data, user preferences, and predefined account settings; and
   Application database 286 for storing local data relevant to the multimodal wellness scores and data rules related thereto, as well as wellness-domain-specific data.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, memory 256, optionally, stores a subset of the modules and data structures identified above. Furthermore, memory 256, optionally, stores additional modules and data structures not described above.

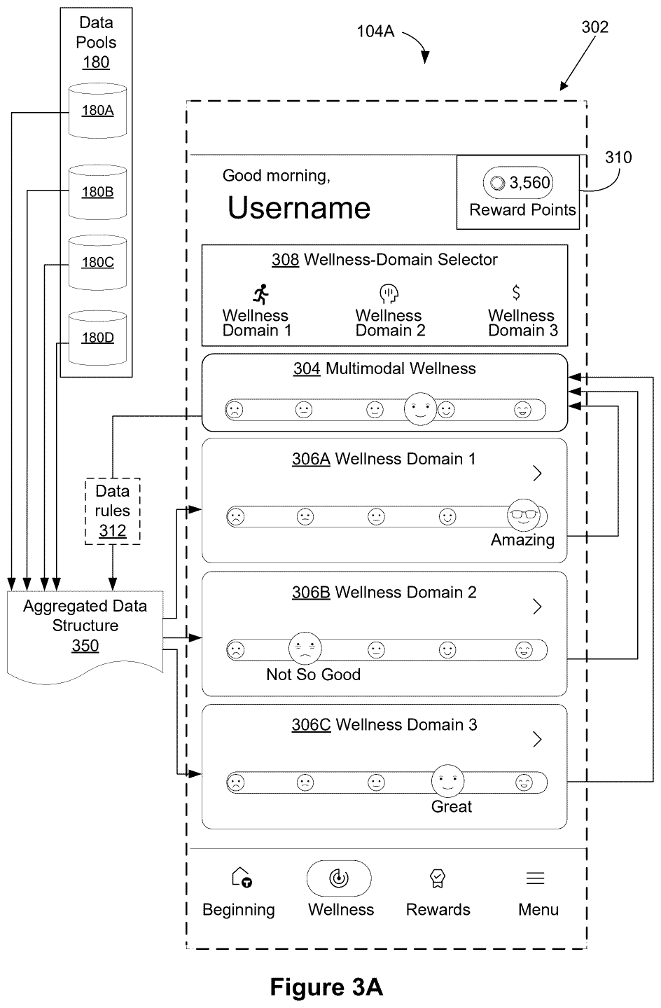

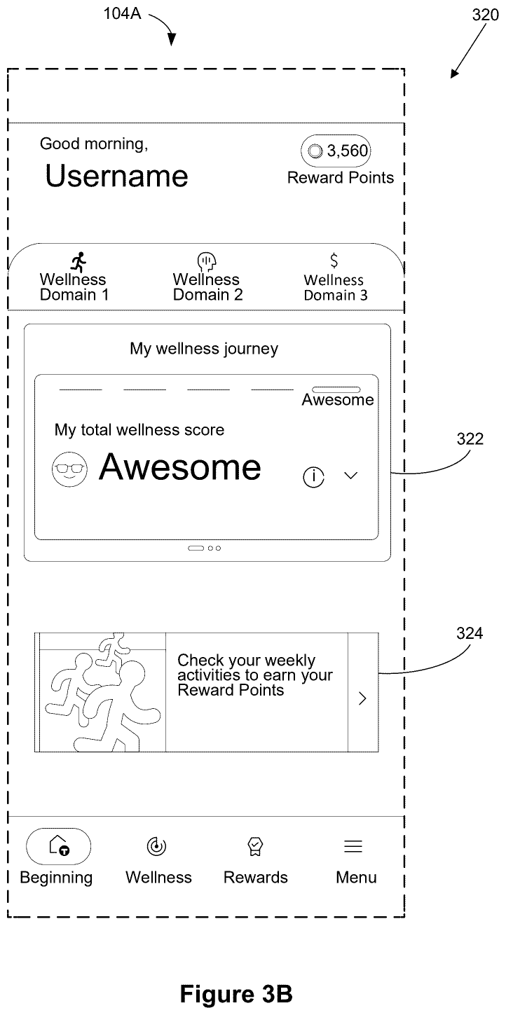

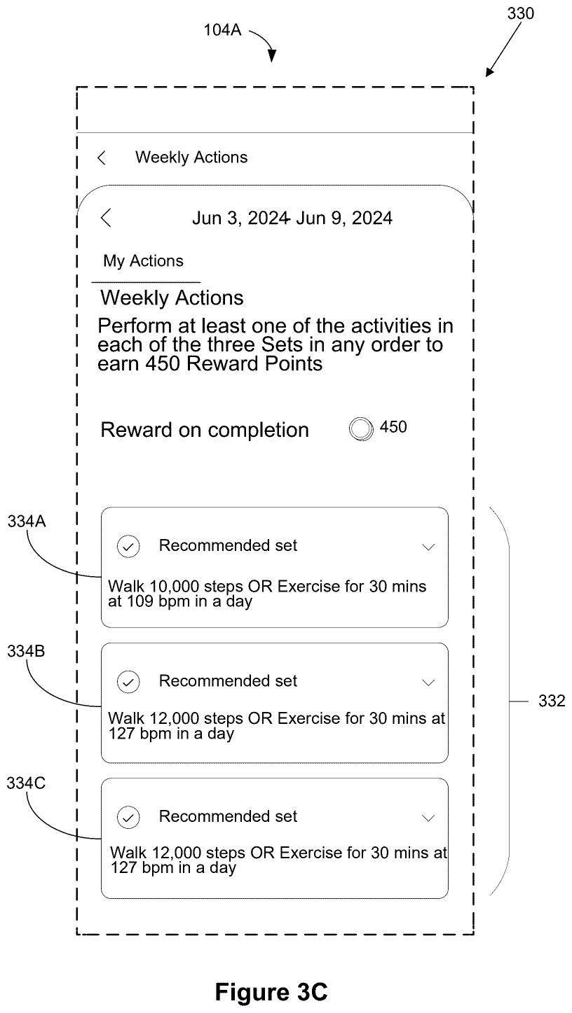

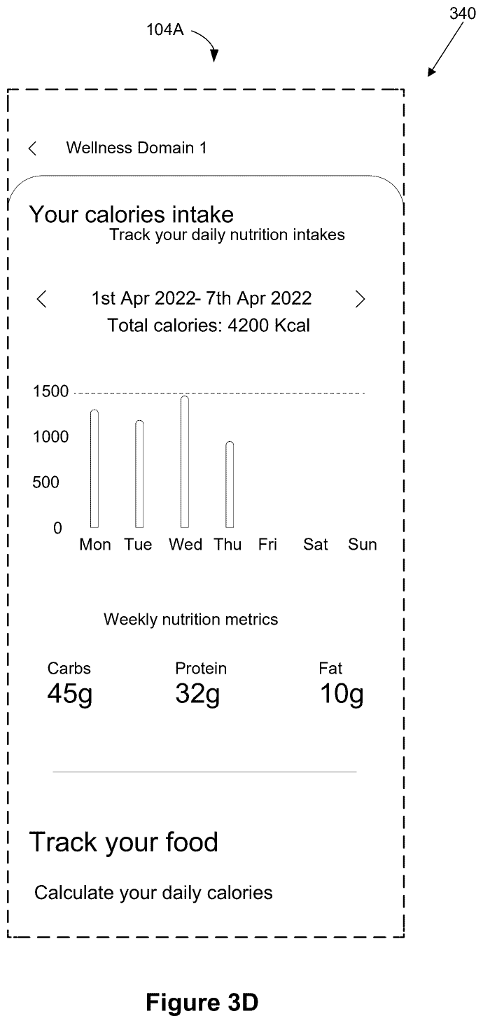

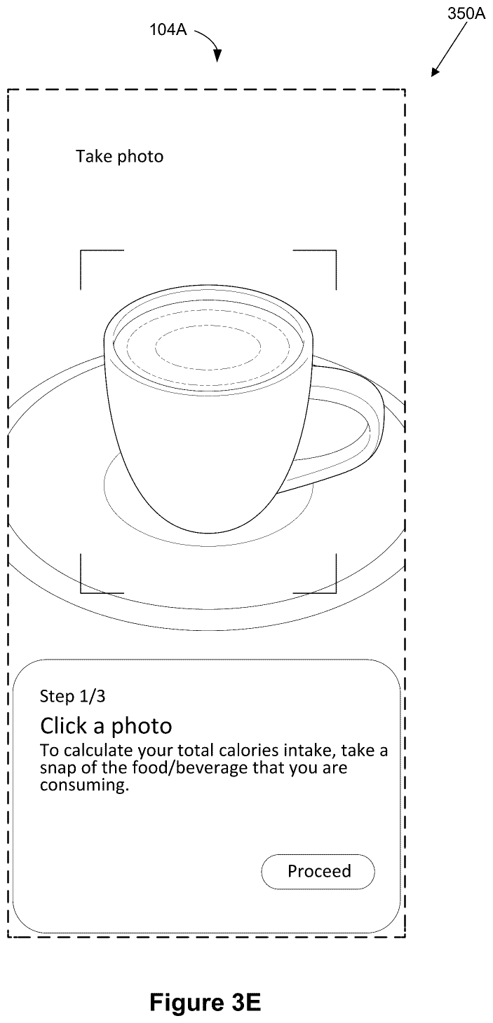

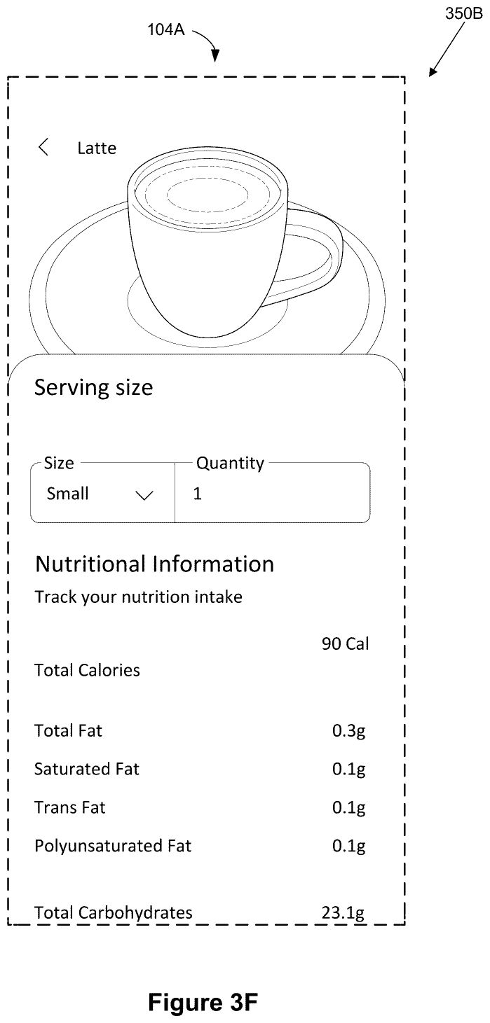

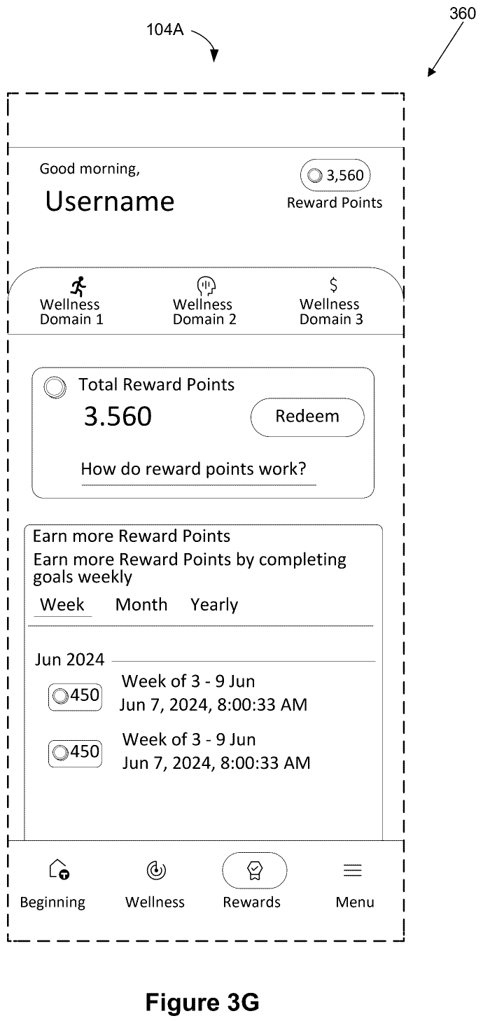

FIGS. 3A to 3G illustrate example user interfaces of a multimodal wellness application, in accordance with some embodiments. For ease of description, the sequence illustrated by FIGS. 3A to 3G will be described with reference to the previously-discussed elements of this disclosure. However, one of skill in the art will appreciate that the operations described herein can be performed by computing systems including additional and/or alternative devices and/or components thereof. The example user interfaces in FIGS. 3A-3G may form an ordered sequence of user interfaces (e.g., displayed in response to user actions).

FIG. 3A illustrates a user interface 302 of the multimodal wellness application 278 described with respect to the computing device 104 in FIG. 2B. The first user interface 302 includes a plurality of user interface elements, including a multimodal wellness indicator 304 (e.g., a wellness progress indicator). In some embodiments, the multimodal wellness indicator 304 indicates an amount of progress that the user has made towards an objective (e.g., a predefined wellness score). In some embodiments, the objective is based on a combination of domain-specific wellness scores based on a plurality wellness domains, which may be predefined by the multimodal wellness application 278 and/or the user 112 (e.g., by configuring preferences of the multimodal wellness application 278).

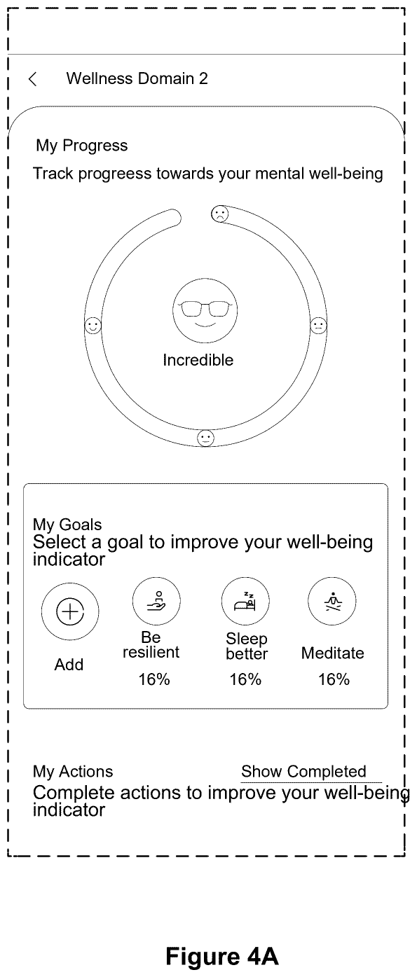

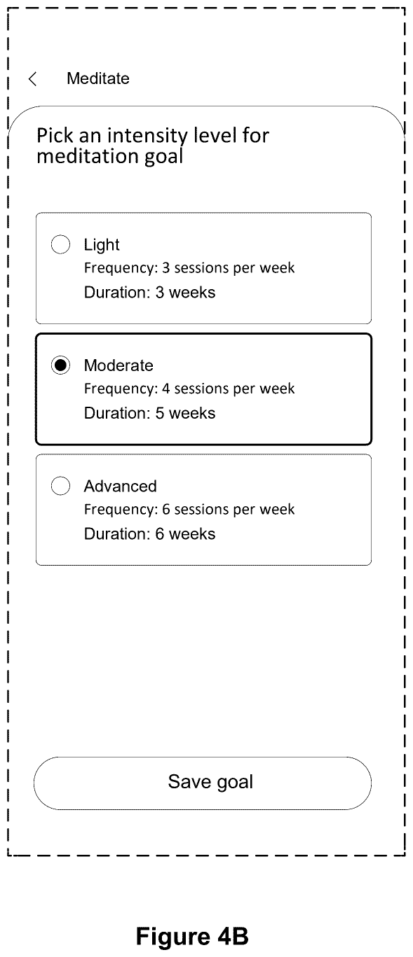

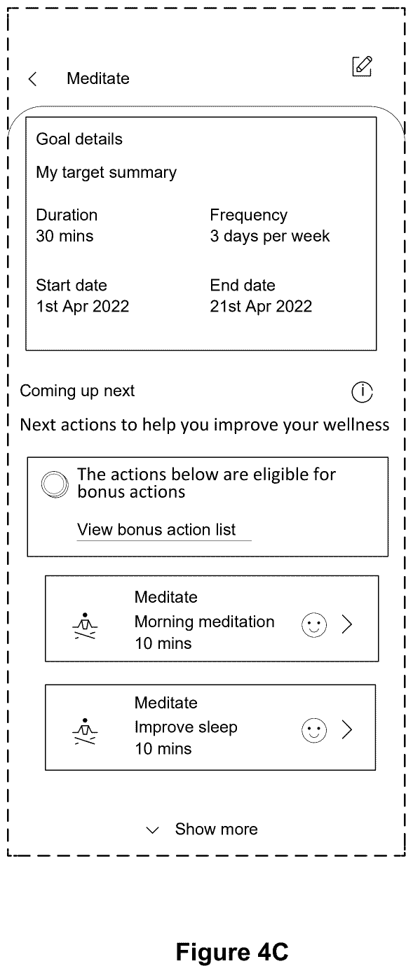

In accordance with some embodiments, the user interface 302 further includes a plurality of wellness-domain-specific user interface elements 306A to 306C. The wellness-domain-specific user interface elements can be associated with specific wellness domains such as physical wellness, mental wellness, and/or fiscal wellness. The user interface 302 further includes a wellness-domain selector 308 for providing user inputs to cause wellness-domain-specific user interfaces to be presented, as described in more detail with respect to FIGS. 4A to 4C.

In accordance with some embodiments, the user interface 302 further includes an incentive score indicator 310 configured to display an amount of incentive points (e.g., reward points, wellness coins, etc.), which may be earned based on progress that the user has made towards the objective indicated by the multimodal wellness indicator 304. In some embodiments, the incentives assigned to one or more respective data rules of the set of data rules 312 is based on the multimodal wellness score of the user 112. In some embodiments, the incentive is the objective that the user progresses towards by performing actions based on the data rules 312.

FIG. 3A also illustrates a data flow diagram showing that data from a plurality of different data pools 180 are provided so that the multimodal wellness application 278 can obtain an aggregation (e.g., an aggregated data structure 350) that can be associated with each respective wellness domain of the plurality of wellness domains to determine a multimodal wellness of the user. In accordance with some embodiments, based on determining the multimodal wellness score indicated by the multimodal wellness indicator 304, a set of data rules (e.g., data rules 312) can be determined. In some embodiments, the data rules 312 can include a set of activities or other actions that the user must perform in order to make progress towards the objective indicated by the multimodal wellness indicator 304.

In some embodiments, the data pools 180 collect respective data in respective wellness domains independently from one another. Each data pool 180 may collect the respective data according to a respective sampling rate. While collecting the respective data, each data pool 180 consolidates the respective data and streams the respective data to the server 106 according to a predefined schedule, in response to a request from the multimodal wellness application 278, or in accordance with a determination that the respective data satisfies a predefined data streaming rule. The multimodal wellness of the user is determined may be determined and updated at the server 106, concurrently with streaming of the data collected by the data pools 180. In some embodiments, at least one of the data pools 180 is associated with a third party, distinct from the server 106 hosting the multimodal wellness application and the computing devices 104 associated with individual users 112.

FIG. 3B illustrates a user interface 320 of the multimodal wellness application 278, including a detail view user interface element 322 of the multimodal wellness indicator 304 shown in FIG. 3A. In accordance with some embodiments, the detail view user interface element 322 is configured to present additional information to the user about the multimodal wellness score determined based on the aggregated data structure 350. In some embodiments, user data of the aggregated data structure 350 can be provided to a machine-learning model associated with the multimodal wellness application 278 to cause natural language text notifications, and/or graphical representations of inferences to be prepared for presentation within the detail view user interface element 322 based on the multimodal wellness data and/or wellness-domain-specific data provided to the machine-learning model.

In accordance with some embodiments, the user interface 320 further includes a banner user interface element 324, which may include automatically- or manually-generated content based on one or more data rules (e.g., activities for the user to perform) determined based on the user's multimodal wellness score. For example, if one of the data rules determined based on the multimodal wellness score is for the user to run a certain distance over a predetermined period of time, then the banner user interface element can provide the user with a prompt to go for a run. In some embodiments, the banner user interface element 324 can include an offer for a higher value of incentive points for performing an activity according to the set of data rules, in order to further incentivize the user to perform the activity or otherwise take action according to a respective data rule of a set of data rules determined based on the multimodal wellness score.

FIG. 3C illustrates a user interface 330 of the multimodal wellness application 278 that includes a data-rule-listing user interface element 332, which presents graphical representations 334 of a plurality of different data rules determined based on applying the aggregated data structure 350 to the plurality of wellness domains to determine the multimodal wellness score indicated by the multimode wellness indicator 304 in FIG. 3A. In some embodiments, one or more of the graphical representations 334 (e.g., graphical representation 334A, graphical representation 334B, and/or graphical representation 334C) are modifiable, such that the user 112 can provide a user input to change a respective graphical representation associated with a particular data rule, without modifying the data rule itself. That is, in some embodiments, a data rule can be more abstract (e.g., burn 500 calories by performing cardio to earn 225 incentive points and/or 0.35% progress towards the objective) than the particular activity or other action that the user sees at the user interface 330 at a particular time. In accordance with some embodiments, when the user 112 modifies a particular graphical representation associated with a particular data rule, that behavior can be stored (e.g., in storage 160).

FIG. 3D illustrates a user interface 340 displaying aspects of a particular wellness domain (e.g., physical health). That is, in some embodiments, the user can perform inputs at the multimodal wellness application 278 to cause user interface elements to be presented that are related to a particular wellness domain of the plurality of wellness domains that were used to determine the multimodal wellness score. For example, the user interface shown in FIG. 3D shows progression of the user with respect to a particular data rule of the set of data rules (e.g., a particular rule related to calorie intake) over a period of time while the data rule has been part of the set of data rules determined based on applying the aggregation to the plurality of wellness domains.

FIGS. 3E to 3F illustrate a set of user interfaces 350A and 350B where the user 112 is capturing sensor data for use by the multimodal wellness application 278 to automatically update progression of the user towards an objective based on a relation between the obtained sensor data and one or more data rules of the set of data rules. That is, in some embodiments, instead of manually updating the multimodal wellness application 278 based on actions that the user performs in accordance with the data rules, the user 112 can also utilize sensor data and/or other data (e.g., third-party data) to cause such actions to automatically be recorded. In some embodiments, a machine-learning model is utilized to analyze the data obtained by the one or more sensors of the computing device (or another computing device).

As shown in FIG. 3E, in some embodiments, the computing device 104 can obtain sensor data corresponding to an activity that the user is performing, such as image data corresponding to a latte that the user is drinking.

As shown in FIG. 3F, the computing device 104 can identify, based on the obtained sensor data, progress corresponding to a respective data rule of the set of data rules; and can automatically apply progress to reward points and/or progress towards the objective defined for the user 112 based on the progress corresponding to the respective data rule.

FIG. 3G shows a user interface 360 that includes user interface elements indicating to the user an amount of incentive points that the user 112 has earned based on performing actions in accordance with the data rules in some embodiments. In some embodiments, when the user 112 either manually or automatically is detected performing actions that correspond to the data rules, adaptive operations can be performed at the multimodal wellness application 278 based on data associated with that particular action or set of actions. For example, if the user has complied with a data rule related to the number of calories that the user has consumed, while also performing a particular number of physical exercises, the multimodal wellness application 278 may infer (e.g., via a machine-learning model) that the multimodal wellness of the user 112 has increased based on performing the actions in accordance with the data rules. Additionally, the user 112 can receive incentive points (e.g., towards a reward provided by a wellness vendor 390A) based on performing the actions. In this way, the techniques described herein improve the incentivization structure for a user 112 to perform actions that benefit their wellness, by accounting for the intrinsic benefit (e.g., the increase in the user's progress towards their wellness objective) while also providing a different benefit to the user based on the incentive points associated with the particular action or set of actions corresponding to the data rules.

FIGS. 4A to 4C illustrate example user interfaces for interacting with a wellness-domain-specific sub-application of a multimodal wellness application, in accordance with some embodiments. Specifically, FIGS. 4A to 4C show that a user can manually configure a particular set of data rules using a set of suggestions provided to the user 112 based on associating the aggregation of the data of the user 112 from the two or more data pools 180 with the plurality of wellness domains (e.g., the plurality of wellness domains associated with the user interface elements 306 in FIG. 3A). The example user interfaces in FIGS. 4A-4C may form an ordered sequence of user interfaces, e.g., displayed in response to user actions.

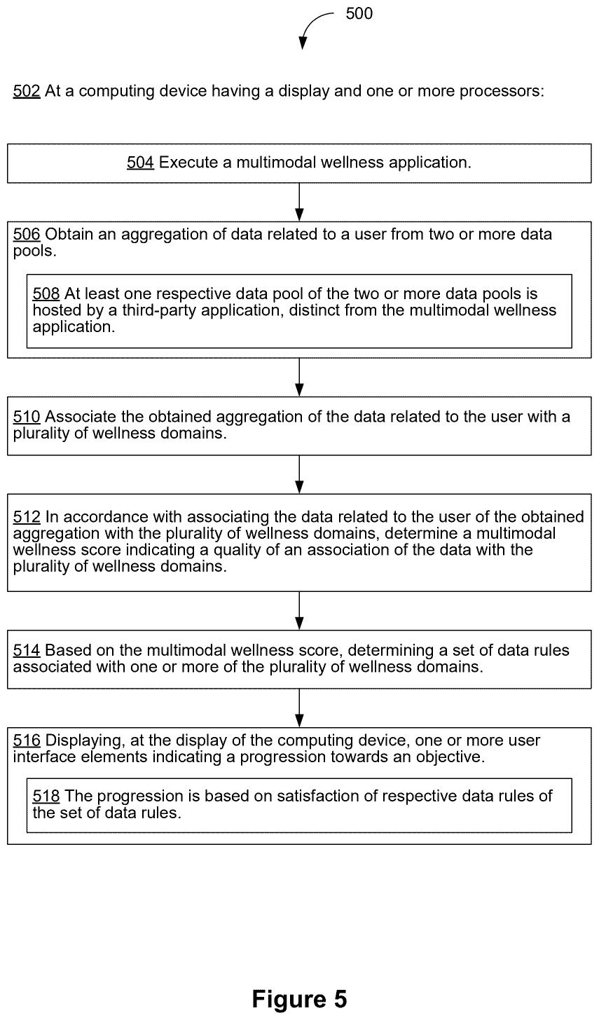

FIG. 5 illustrates a flow diagram of an example method 500 for managing data, in accordance with some embodiments. In an example, the method is implemented for real-time prediction and visualization of wellness-related data. For convenience, the method 500 is described as being implemented by the computing device 104. Method 500 is, optionally, governed by instructions that are stored in a non-transitory computer-readable storage medium and that are executed by one or more processors of the computer system. Each of the operations shown in FIG. 5 may correspond to instructions stored in a computer memory or non-transitory computer readable storage medium (e.g., memory 206 in FIG. 2A and/or 256 in FIG. 2B). The non-transitory computer-readable storage medium may include a magnetic or optical disk storage device, solid state storage devices such as Flash memory, or other non-volatile memory device or devices. The instructions stored on the computer readable storage medium may include one or more of: source code, assembly language code, object code, or other instruction format that is interpreted by one or more processors. Some operations in method 500 may be combined and/or the order of some operations may be changed.

(A1) In some embodiments, the method 500 is performed at a computing device having a display and one or more processors (operation 502). For example, the operations of the method 500 may be performed at the desktop computer 104A, the mobile phone 104D, and/or the application server 106A. In some embodiments, portions of the method 500 and/or related operations for facilitating performance of the method 500 can be performed by a combination of computing devices.

The computing device executes (operation 504) a multimodal wellness application. For example, FIGS. 3A to 3G illustrate an example of a multimodal wellness application being executed at the mobile phone 104D.

The computing device obtains (operation 506) (e.g., streams, downloads) an aggregation of data related to a user (e.g., (i) fiscal behavior, (ii) healthcare behavior, and/or (iii) customer behavior) from two or more data pools (e.g., managed data environments comprising data associated with a plurality of users 112). For example, FIG. 1 shows the API gateway 110 communicating with the servers 106A and 106B to receive data from the two or more of the data pools 180 (e.g., a data pool 180A that is associated with the application server 106A, and another data pool 180B that is associated with one of the third-party servers 106B).

At least one respective data pool of the two or more data pools is hosted by a third-party application, distinct from the multimodal wellness application (operation 508). For example, as discussed above with respect to operation 606, the data pool 180B may be associated with (e.g., hosted by) a third-party server 106B of one or more of the third-party systems 190 (e.g., the healthcare system 198).

The computer system associates (operation 510) the obtained aggregation of the data related to the user with a plurality of wellness domains (e.g., predefined wellness domains, such as a physical health wellness domain, a mental health wellness domain, and fiscal health wellness domain). For example, the aggregation may be associated with two or more of the wellness domains depicted by the wellness domain user interface elements 3xx shown in FIG. 3A.

In accordance with associating the data related to the user of the obtained aggregation with the plurality of wellness domains, the computer system determines (operation 512) (e.g., using a scoring algorithm) a multimodal wellness score indicating a quality of an association of the data with the plurality of wellness domains.

Based on the multimodal wellness score (e.g., an overall condition indicator), the computer system determines (operation 514) a set of data rules (e.g., action instructions, activities, criteria) associated with one or more of the plurality of wellness domains.

The computer system displays (operation 516), at the display of the computing device, one or more user interface elements indicating a progression towards an objective (e.g., an incentive, a reward).

The progression is based on satisfaction of respective data rules of the set of data rules (operation 518). For example, as the user performs the activities depicted by the user interface elements 3xx in FIG. 3C, which associated with respective data rules of the set of data rules, the progression of one or more wellness domains of the user may increase, which can cause a corresponding increase in progress towards the objective (e.g., reward points, an increased multimodal wellness score, etc.).

(A2) In some embodiments of A1, the method 500 further includes, based on determining that a respective data rule of the set of data rules has been satisfied by the user, dynamically updating the multimodal wellness score based on a type of the respective data rule and an updated progress towards the objective. For example, in accordance with performing one of the actions described by the representations 334 in FIG. 3C, an aggregated data structure 350 associated with the user can be updated, and re-applied to the plurality of wellness domains represented by the wellness-domain-specific user interface elements 306 shown in FIG. 3A.

In some embodiments, in accordance with dynamically updating the multimodal wellness score, the computing device 104 can be caused to automatically update the set of data rules. In other words, as the user causes the multimodal wellness score to change by satisfying the data rules, the set of data rules can be modified to account for the updated multimodal wellness score. In some embodiments, a respective data rule of the set of data rules 312 can have a particular guaranteed duration that it will be available for the user perform a set of actions that satisfies the respective data rule (and earns the incentive points associated with the data rule).

(A3) In some embodiments of A1 or A2, respective amounts of progress associated with satisfaction of a respective data rules of the set of data rules are weighted based on domain-specific components of the multimodal wellness score. For example, if a mental health domain component of the multimodal wellness score is relatively low compared to other domain-specific components (e.g., the wellness domain 2 as represented in FIG. 3B has a least amount of progress among the plurality of wellness domains indicated by the wellness-domain-specific user interface elements 306 in FIG. 3A), then a particular data rule related to mental health of the set of data rules could be weighted such that the particular data rule is associated with a larger amount of progress than another particular data rule related to physical health.

(A4) In some embodiments of any one of A1 to A3, the method 500 further includes, after the user has completed progression toward the objective, in response to a user request for a content item (e.g., a reward, such as a digital content item or a physical content item), selecting a subset of content items from a plurality of content items identified as selectable by the user, based on the multimodal wellness score. In some embodiments, the method 500 further includes displaying the subset of content items on the display of the computing device, and (iii) in response to a user selection of a respective displayed content item of the subset of content items, adjusting the progression towards the objective based on a reward credit of the selected respective displayed content item.

(A5) In some embodiments of any one of A1 to A4, the set of data rules is determined using a model (e.g., an artificial intelligence model) customized for the user, based on a behavioral history of the user.

(A6) In some embodiments of any one A1 to A5, the method 500 further includes, after identifying the set of data rules, in accordance with a determination that the user has not satisfied a respective data rule of the set of data rules within a performance threshold duration, providing a notification to the user related to the respective data rule.

(A7) In some embodiments of any one of A1 to A6, an amount of progress associated with satisfaction of a respective data rule of the set of data rules is weighted based on a corresponding wellness-domain-specific aspect of the multimodal wellness score.

(A8) In some embodiments of any one of A1 to A7, the method 500 further includes: (i) obtaining, at the computing device, sensor data (e.g., image data, health data) corresponding to an activity that the user is performing; (ii) identifying, based on the obtained sensor data, progress corresponding to a respective data rule of the set of data rules; and (iii) updating the progression towards the objective based on the identified progress from the sensor data (e.g., extracting nutrition data from one or more images provided by an electronic device associated with the first user account). For example, the user can capture an image of a coffee that the user is drinking, and the image can be used to update progress of completion of a nutritional goal assigned to the user.

(A9) In some embodiments of any one of A1 to A8, the method 500 further includes presenting a plurality of wellness-balance user interface elements at the display of the computing device, wherein each of the wellness-balance user interface elements correspond to a respective wellness domain of the plurality of wellness domains.

(A10) In some embodiments of A9, the method 500 further includes responsive to a user input directed to a respective wellness-balance user interface elements of the plurality of wellness-balance user interface elements, presenting a user interface corresponding to a particular wellness domain of the plurality of wellness domains, wherein the user interface corresponding to the particular wellness domain includes additional information related to the particular wellness domain.

(A11) In some embodiments of A10, the method 500 further includes while the user interface corresponding to the particular wellness domain is being displayed, providing one or more user interface elements for configuring a respective data rule based on the multimodal wellness score.

(A12) In some embodiments of any one of A1 to A11, a machine-learning model (e.g., a neural network, such as a large-language model, machine-learning model 125 shown in FIG. 1) is electronically coupled with the multimodal wellness application. In some embodiments, the machine-learning model is configured to determine one or more structural features of the aggregation of the data related to the user based on one or more features of the data received from the two or more data pools.

(A13) In some embodiments of A12, the machine-learning model is further configured to generate a display element to display at the computing device (e.g., in conjunction with the one or more user interface elements indicating the progression towards the objective) based on applying a respective data rule of the set of data rules to a natural language. For example, the machine-learning model 125 shown in FIG. 1 may be configured to generate text for displaying within the detail view user interface element 322 shown in FIG. 3B (e.g., a textual phrase that coaches the user to perform actions more efficiently in accordance with the set of data rules).

(A14) In some embodiments of any one of A1 to A13, the method 500 further includes identifying one or more banner content items to present to the user in conjunction with displaying the one or more user interface elements indicating the progression of the user towards the objective. For example, the banner content item 324 shown in FIG. 3B can be configured to present banner content based on information from the aggregated data structure 350, the determined wellness scores, and/or the set of data rules determined based on the determined wellness scores.

(A15) In some embodiments of any one of A1 to A14, the method 500 includes limiting a request rate to the two or more data pools based on settings of one or more of third-party providers. In some embodiments, API keys are dynamically rotated between requests based on the settings, or other settings of a different provider. For example, the API gateway 110 shown in FIG. 1 may determine a particular request rate for each of the plurality of servers that the multimodal wellness application is in electronic communication with.

(A16) In some embodiments of A15, the request rate is further based on one or more optimal fetch intervals, the one or more optimal fetch intervals determined based on a data change frequency of the respective data pools of the two or more data pools. Thus, the intelligent data retrieval techniques described herein can be managed for performance and/or cost of one or more components of the multimodal wellness system.

(A17) In some embodiments A16, the method 500 further includes, in accordance with fetching data at one of the one or more optimal fetch intervals, generating a timestamp that includes (i) a moment of data change, and (ii) a contextual data signature comprising addition information about an aspect of the data change (e.g., to improve traceability and analytics). For example, when the API gateway 110 shown in FIG. 1 requests information from a particular server of the servers 106, it can obtain information indicating how long the data is validated for (e.g., how long the data can be used to determine the multimodal wellness score before another request must be made).

(A18) In some embodiments of any one of A1 to A17, each respective application of the different third-party applications and the multimodal wellness application include separate authentication techniques for accessing data from the respective applications, and the method further includes generating a token associated with a different authentication technique than any of the respective authentication techniques of the respective applications of the third-party application or the multimodal wellness application, the token configured to cause access to be provided to the aggregation of the two or more data pools. In some embodiments, data synchronization can further be used to flag unusual requests as potential security threats.

(B1) In some embodiments, a server (e.g., the application server 106A in FIG. 1) is provided. The server includes one or more processors and memory that includes instructions which, when executed by the one or more processors, cause the processors to perform a method of any of A1 to A18.

(C1) In some embodiments, a non-transitory computer-readable storage medium (e.g., a non-transitory computer-readable storage medium associated with the multimodal wellness application 278), which, when executed by the one or more processors, cause the one or more processors to perform a method of any of A1 to A18.

It should be understood that the particular order in which the operations in FIG. 5 have been described are merely exemplary and are not intended to indicate that the described order is the only order in which the operations could be performed. One of ordinary skill in the art would recognize various ways to predict and visualize a data trend. Additionally, it should be noted that details of other processes described above with respect to FIGS. 1-4C are also applicable in an analogous manner to method 500 described above with respect to FIG. 5. For brevity, these details are not repeated here.

Some implementations associated with different aspects of the computing system 100 are further discussed as follows:

### Multimodal Data Collection

In some embodiments, the multimodal wellness system 102 (FIG. 1) imports, from one of the data pools, physical activity data of a first user account from one or more third-party activity applications distinct from the multi-modal wellness application 230 (FIG. 2A). The one or more third-party activity applications may be integrated with the multi-modal wellness application 230 via respective application programming interfaces (APIs). In some embodiments, the APIs utilizes cryptographic techniques in an authorization process, ensuring that even if tokens are intercepted, the physical activity data remain unusable. In some embodiments, the APIs may apply API keys that are dynamically rotated, and employ multi-factor validation for access, thereby reducing vulnerability from static keys and ensuring prolonged data protection. In some embodiments, adaptive rate-limiting, is applied where a request threshold varies based on traffic patterns, ensuring optimal data flow without overburdening the data pools 180 (FIG. 1).

In some embodiments, less than all data collected by a data pool 180 (e.g., pool 180A) is streamed to the server 106A associated with the multimodal wellness system 102. A subset of data is selected by the data pool 180 based on a data pooling method. The data pooling method is dynamically and automatically adjusted. In accordance with the data pooling method, an incremental fetch may be applied to select changed data for streaming to the server 106A, and data fetch intervals may be adjusted based on data change frequencies. In some embodiments, a data item may be provided to the server 106A jointly with one or more of a timestamp, contextual data signatures, preliminary data analysis indicating a nature of a data change.

In some embodiments, the server 106A applies machine learning to process data collected from a new data pool 180, and trains a data processing model automatically to recognize a data structure used by the data collected from the new data pool 180.

In some embodiments, the server 106A validates the data collected from the data pool 180 using probabilistic data matching. In some embodiments, even non-exact data entries get validated against possible matches. A matching probability may be determined and compared with a threshold to determine whether to validate a data entry.

### Data Integration, Duplication, and Synchronization

In some embodiments, a schema applied in the multimodal wellness system 102 is dynamic, and employs a modular structure allowing components to adapt and evolve as the nature of imported data changes.

In some embodiments, the multimodal wellness system 102 (FIG. 1) employs differential backups, storing only changes since the last backup, combined with AI-driven recovery prediction, drastically reducing restoration times.

In some embodiments, while conventional algorithms rely on key attributes, the multimodal wellness system 102 employs a neural network-driven approach, detecting nuanced data duplicates even when key attributes diverge.

In some embodiments, the multimodal wellness system 102 employs behavioral analytics, flagging unusual request patterns as potential security threats.

In some embodiments, the multimodal wellness system 102 processes events with a context-aware mechanism, adapting actions based on current system states and past event patterns.

Data Extraction from User Behavior and Images

In some embodiments, the multimodal wellness system 102 uses specialized OCR libraries and cloud-based OCR APIs, finely tuned for versatility and accuracy across a diverse range of images and conditions. In some embodiments, image preprocessing integrates machine learning-driven noise reductions and contrast adjustments to optimize low-quality images for data extraction.

In some embodiments, the multimodal wellness system 102 accurately identifies and categorizes relevant entities, drawing from user behavior and context. In some embodiments, the system 102 employs deeply integrated natural language processing (NLP) models that are fine-tuned to discern ambiguities and nuances in the data.

In some embodiments, EXIF data serves as a tool to contextualize data extraction, drawing correlations from timestamps and geolocations. In some embodiments, geocoding extends beyond mere latitude-longitude conversions to amalgamate wellness insights, turning coordinates into a repository of localized habits or insights.

### Acquisition and Integration of Fiscal Data

In some embodiments, leveraging API integrations with reputable banking and financial institutions, the multimodal wellness system 102 meticulously extracts, encrypts, and processes transactional and account data. To illuminate the sophisticated interplay between financial health, physical activity, nutritional markers, and mental health indicators, proprietary algorithms sift through data, resulting in a holistic snapshot of a user's financial well-being.

In some embodiments, the multimodal wellness system 102 runs on a multifaceted algorithm that seamlessly merges quantitative financial metrics, such as expenses, savings, and investments, with qualitative data extracted from the physical, mental, and nutritional health modules. Through weighted analytics and neural network evaluations, this amalgamation showcases the role of financial health in the broader context of well-being.

In some embodiments, central to the multimodal wellness system's design is a dynamic database updating framework. It continuously assimilates real-time data inputs from health wearables, mobile trackers, mental well-being apps, and financial institutions. A robust real-time, event-driven architecture ensures that the overall condition indicator is consistently recalibrated as new data streams in, supported by high-frequency polling and webhooks for data integrity.

In some embodiments, the multimodal wellness system 102 is powered by a composite AI model trained on vast datasets. This model deciphers the nuanced relationships between financial behaviors, physical health markers (e.g., activity levels, nutritional intake), and crucial mental health indicators (e.g., stress, mood, cognitive function). This model, fine-tuned through continuous machine learning, adapts to individual user profiles, enhancing its correlative and predictive capabilities.

### Dynamic Score Generation

In some embodiments, a dynamic scoring algorithm provides an adaptive and multifaceted assessment of individual wellness, covering not just financial health but also physical and mental well-being. Utilizing machine learning, incremental data fetching, and advanced analytics, the algorithm continually updates and personalizes wellness scores across multiple domains. It offers a 360-degree view of an individual's life, taking into account variables from income and debt to physical fitness levels and mental resilience.

In some embodiments, upon user initiation, the multimodal wellness system 102 triggers a context-aware request handler that rapidly interfaces with the user's stored financial and overall condition data. By integrating with state-of-the-art database management systems and utilizing indexed searches, the multimodal wellness system 102 efficiently assesses eligibility and appropriateness for potential rewards.

In some embodiments, the multimodal wellness system 102 boasts a deep learning neural network trained on past user preferences, behaviors, and current overall condition indicators. By accessing the vast repository of available rewards, the AI algorithm dynamically curates a subset tailored to the user's current state, ensuring relevance and enhancing the likelihood of positive user engagement.

### Information Presentation

In some embodiments, a UI/UX module integrated with the main platform ensures that the curated reward options are presented in an aesthetically pleasing, responsive, and intuitive manner. Leveraging the capabilities of modern frontend frameworks and adhering to best design principles, the multimodal wellness system 102 ensures optimal visual performance across varied electronic devices.

In some embodiments, following user selection, an event-driven mechanism triggers the redemption process. Simultaneously, it may activate the overall condition adjuster module. By accessing predefined reward credit parameters and cross-referencing them with the user's profile, the multimodal wellness system 102 recalibrates the overall condition indicator. This is achieved through a combination of weighted adjustments, real-time data synchronization, and transactional database operations.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Additionally, it will be understood that, although the terms “first,” “second,” etc. may be used herein to describe various elements, these elements should not be limited by these terms. These terms are only used to distinguish one element from another.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

Although various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages can be implemented in hardware, firmware, software, or any combination thereof.

What is claimed is:

1. A method, comprising:
at a computing device having a display and one or more processors:
executing a multimodal wellness application;
obtaining an aggregation of data related to a user of two or more data pools, wherein at least one respective data pool of the two or more data pools is hosted by a third-party application, distinct from the multimodal wellness application;
associating the obtained aggregation of the data related to the user with a plurality of wellness domains;
in accordance with associating the data related to the user of the obtained aggregation with the plurality of wellness domains, determining a multimodal wellness score indicating a quality of an association of the data with the plurality of wellness domains;
based on the multimodal wellness score, determining a set of data rules associated with one or more of the plurality of wellness domains; and
displaying, at the display of the computing device, one or more user interface elements indicating a progression towards an objective, wherein the progression is based on satisfaction of respective data rules of the set of data rules.

2. The method of claim 1, further comprising:
based on determining that a respective data rule of the set of data rules has been satisfied by the user, dynamically updating the multimodal wellness score based on a type of the respective data rule and an updated progress towards the objective; and
in accordance with dynamically updating the multimodal wellness score, updating the set of data rules.

3. The method of claim 1, wherein respective amounts of progress associated with satisfaction of a respective data rules of the set of data rules are weighted based on domain-specific components of the multimodal wellness score.

4. The method of claim 1, further comprising:
after the user has completed progression toward the objective, in response to a user request for a content item, selecting a subset of content items from a plurality of content items identified as selectable by the user, based on the multimodal wellness score;
displaying the subset of content items on the display of the computing device; and
in response to a user selection of a respective displayed content item of the subset of content items, adjusting the progression towards the objective based on a reward credit of the selected respective displayed content item.

5. The method of claim 1, wherein the set of data rules is determined using a model customized for the user, based on a behavioral history of the user.

6. The method of claim 1, further comprising:
after identifying the set of data rules, in accordance with a determination that the user has not satisfied a respective data rule of the set of data rules within a performance threshold duration, providing a notification to the user related to the respective data rule.

7. The method of claim 1, wherein an amount of progress associated with satisfaction of a respective data rule of the set of data rules is weighted based on a corresponding wellness-domain-specific aspect of the multimodal wellness score.

8. The method of claim 1, further comprising:
obtaining, at the computing device, sensor data corresponding to an activity that the user is performing;
identifying, based on the obtained sensor data, progress corresponding to a respective data rule of the set of data rules; and
updating the progression towards the objective based on the identified progress from the sensor data.

9. The method of claim 1, further comprising:
presenting a plurality of wellness-balance user interface elements at the display of the computing device, wherein each of the wellness-balance user interface elements correspond to a respective wellness domain of the plurality of wellness domains.

10. The method of claim 9, further comprising:
responsive to a user input directed to a respective wellness-balance user interface elements of the plurality of wellness-balance user interface elements, presenting a user interface corresponding to a particular wellness domain of the plurality of wellness domains, wherein the user interface corresponding to the particular wellness domain includes additional information related to the particular wellness domain.

11. The method of claim 10, further comprising:
while the user interface corresponding to the particular wellness domain is being displayed, providing one or more user interface elements for configuring a respective data rule based on the multimodal wellness score.

12. A server system, comprising:
one or more processors; and
memory having instructions stored thereon, which when executed by the one or more processors cause the processors to perform operations including:
executing a multimodal wellness application;
obtaining an aggregation of data related to a user of two or more data pools, wherein at least one respective data pool of the two or more data pools is hosted by a third-party application, distinct from the multimodal wellness application;
associating the obtained aggregation of the data related to the user with a plurality of wellness domains;
in accordance with associating the data related to the user of the obtained aggregation with the plurality of wellness domains, determining a multimodal wellness score indicating a quality of an association of the data with the plurality of wellness domains;
based on the multimodal wellness score, determining a set of data rules associated with one or more of the plurality of wellness domains; and
displaying, at a display of a computing device, one or more user interface elements indicating a progression towards an objective, wherein the progression is based on satisfaction of respective data rules of the set of data rules.

13. The server system of claim 12, wherein a machine-learning model is electronically coupled with the multimodal wellness application, the machine-learning model configured to:
determine one or more structural features of the aggregation of the data related to the user based on one or more features of the data received from the two or more data pools.

14. The server system of claim 13, wherein the machine-learning model is further configured to generate a display element to display at the computing device based on applying a respective data rule of the set of data rules to a natural language.

15. The server system of claim 12, the memory further comprising instructions for:
identifying one or more banner content items to present to the user in conjunction with displaying the one or more user interface elements indicating the progression of the user towards the objective.

16. A non-transitory computer-readable storage medium, having instructions stored thereon, which when executed by one or more processors cause the one or more processors to perform operations comprising:
executing a multimodal wellness application;
obtaining an aggregation of data related to a user of two or more data pools, wherein at least one respective data pool of the two or more data pools is hosted by a third-party application, distinct from the multimodal wellness application;
associating the obtained aggregation of the data related to the user with a plurality of wellness domains;
in accordance with associating the data related to the user of the obtained aggregation with the plurality of wellness domains, determining a multimodal wellness score indicating a quality of an association of the data with the plurality of wellness domains;
based on the multimodal wellness score, determining a set of data rules associated with one or more of the plurality of wellness domains; and
displaying, at a display of a computing device, one or more user interface elements indicating a progression towards an objective, wherein the progression is based on satisfaction of respective data rules of the set of data rules.

17. The non-transitory computer-readable storage medium of claim 16, further comprising instructions for:
limiting a request rate to the two or more data pools based on settings of one or more third-party providers.

18. The non-transitory computer-readable storage medium of claim 17, wherein the request rate is further based on one or more optimal fetch intervals, the one or more optimal fetch intervals determined based on a data change frequency of the respective data pools of the two or more data pools.

19. The non-transitory computer-readable storage medium of claim 18, further comprising instructions for:
in accordance with fetching data at one of the one or more optimal fetch intervals, generating a timestamp that includes (i) a moment of data change, and (ii) a contextual data signature comprising addition information about an aspect of the data change.

20. The non-transitory computer-readable storage medium of claim 16, wherein each respective application of the third-party application and the multimodal wellness application include separate authentication techniques for accessing data from the respective applications, the non-transitory computer-readable storage medium further comprising for:
generating a token associated with a different authentication technique than any of the respective authentication techniques of the respective applications of the third-party application or the multimodal wellness application, the token configured to cause access to be provided to the aggregation of the two or more data pools.
